(19) United States
(12) Patent Application Publication
(10) Pub. No.: US 2008/0213320 A1
(43) Pub. Date: Sep. 4, 2008

(54) Compositions for treatment of gastro-esophageal reflux disorders

(75) Inventors: Mayer Eisenstein, Chicago, IL (US); Jeremy B. Eisenstein, Evanston, IL (US)

Correspondence Address: BRINKS HOFER GILSON & LIONE, P.O. BOX 10395, CHICAGO, IL, 60610, US

(73) Assignee: Jeremy B. Eisenstein

(21) Appl. No.: 11/825,005

(22) Filed: Jul. 3, 2007

Related U.S. Application Data
(60) Provisional application No. 60/904,291, filed on Mar. 1, 2007.

(51) Int. Cl. A61K 38/54; A61K 36/00; A61K 39/00; A61K 9/00; A61P 1/14

(52) U.S. Cl. 424/400; 424/725; 424/93.45; 424/94.2

(57) ABSTRACT

The present invention provides compositions for treatment of gastro-esophageal reflux disorders. The compositions include at least two of the following: (i) one or more digestive enzymes; (ii) one or more probiotics; and (iii) stevia. Also provided are processes for preparing the compositions useful for treatment of gastro-esophageal reflux disorders, and methods of treating subjects against gastro-esophageal reflux disorders, which include administering to a subject a therapeutically effective amount of the compositions of the present invention.

### CROSS-REFERENCE TO RELATED APPLICATIONS

This invention claims priority to U.S. Provisional Patent Application Ser. No. 60/904,291, filed Mar. 1, 2007.

### FIELD OF THE INVENTION

This invention relates to compositions and methods for treatment of gastro-esophageal reflux disorders.

### BACKGROUND OF THE INVENTION

Gastro-esophageal reflux disorders, including gastro-esophageal reflux disease (GERD), acid indigestion, over-indulgence of food or drink, acid stomach, sour stomach, waterbrash/regurgitation, heartburn, such as episodic heartburn, nocturnal heartburn, meal-induced heartburn and dyspepsia, affect millions of people at least once a week. Heartburn is the most common symptom of gastro-esophageal reflux disorders. The sensation of heartburn is caused by exposure of the lower esophagus to the acidic contents of the stomach. Normally, the lower esophageal sphincter separating the stomach from the esophagus is supposed to contract to prevent this from occurring. If the sphincter relaxes for any reason (as normally occurs during swallowing), stomach contents, mixed with gastric acid, can return into the esophagus. This return is also known as reflux, which results in a painful or burning sensation in the esophagus, just below the breastbone caused by regurgitation of gastric acid. The pain often rises in the chest and may radiate to the neck, throat, or angle of the jaw.

It is important to know how to treat the gastro-esophageal reflux disorders. Restricting and managing diet can be helpful, since many sufferers of heartburn or related gastro-esophageal disorders can link their Gastro-esophageal reflux disorders, including gastro-esophageal reflux disease (GERD), acid indigestion, over-indulgence of food or drink, acid stomach, sour stomach, waterbrash/regurgitation, heartburn, such as episodic heartburn, nocturnal heartburn, meal-induced heartburn and dyspepsia, affect millions of people at least once a week. Heartburn is the most common symptom of gastro-esophageal reflux disorders. The sensation of heartburn is caused by exposure of the lower esophagus to the acidic contents of the stomach. Normally, the lower esophageal sphincter separating the stomach from the esophagus is supposed to contract to prevent this from occurring. If the sphincter relaxes for any reason (as normally occurs during swallowing), stomach contents, mixed with gastric acid, can return into the esophagus. This return is also known as reflux, which results in a painful or burning sensation in the esophagus, just below the breastbone caused by regurgitation of gastric acid. The pain often rises in the chest and may radiate to the neck, throat, or angle of the jaw.

It is important to know how to treat the gastro-esophageal reflux disorders. Restricting and managing diet can be helpful, since many sufferers of heartburn or related gastro-esophageal disorders can link their symptoms to specific foods. For individuals who suffer from chronic heartburn, whether it is caused by gastro-esophageal reflux disorders or some other digestive disorder, the treatment can range from lifestyle change to surgery in severe cases that do not respond to any other treatment.

Prescription medications for gastro-esophageal reflux disorders include proton pump inhibitors (such as esomeprazole and omeprazole) and acid reducers, also known as H2 blockers (such as nizatidine and cimetidine). However, some of the medications for treatment of gastro-esophageal reflux disorders have undesired side effects. Some of the reported side effects of Nexium®, Prevacid®, Prilosac®, and Tagamet® are headache, diarrhea, upset stomach, gas, stomach pain, constipation, and dry mouth.

### BRIEF SUMMARY

The present invention provides compositions for treatment of a gastro-esophageal reflux disorders. The compositions include at least two of the following: (i) one or more digestive enzymes; (ii) one or more probiotic compounds (probiotics); and (iii) stevia.

The compositions may include one or more digestive enzymes, including but not limited to peptidase, protease, bromelain, papain, amylase, lactase, lipase, and hemicellulase. The compositions may include about 5,000 HUT to about 30,000 HUT peptidase. The compositions may include about 15,000 HUT to about 50,000 HUT protease. The compositions may include about 100 GDU to about 400 GDU bromelain. The compositions may include about 100,000 PU to about 200,000 PU papain. The compositions may include about 5,000 DU to about 25,000 DU amylase. The compositions may include about 500 ALU to about 1,500 ALU lactase. The compositions may include about 2,500 LU to about 8,000 LU lipase. The compositions may include about 50 HCU to about 250 HUC hemicellulase. The compositions may include one or more probiotics, including but not limited to Lactobacillus acidophilus, Lactobacillus casei Lactobacillus plantarum, Lactobacillus rhamnosus, Bifidobacterium breve, and Bifidobacterium longum. In one example, the compositions include about 2,500,000,000 CFU of probiotics. The compositions may include stevia. In one example, the compositions include about 10 mg to about 20 mg stevia.

Gastro-esophageal reflux disorders that can be treated using the compositions and methods of the present invention include but are not limited to: gastro-esophageal reflux disease (GERD), acid indigestion, over-indulgence of food or drink, acid stomach, sour stomach, waterbrash/regurgitation, heartburn, and dyspepsia.

The present invention provides oral compositions for treatment of gastro-esophageal reflux disorders. The oral compositions include: a) a vehicle for oral delivery; b) a water soluble portion; and c) a coating layer including at least two of the following: (i) one or more digestive enzymes; (ii) one or more probiotics; and (iii) stevia.

The oral compositions may include one or more digestive enzymes, including but not limited to peptidase, protease, bromelain, papain, amylase, lactase, lipase, and hemicellulase. The oral compositions may include about 5,000 HUT to about 30,000 HUT peptidase. The oral compositions may include about 15,000 HUT to about 50,000 HUT protease. The oral compositions may include about 100 GDU to about 400 GDU bromelain. The oral compositions may include about 100,000 PU to about 200,000 PU papain. The oral compositions may include about 5,000 DU to about 25,000 DU amylase. The oral compositions may include about 500 ALU to about 1,500 ALU lactase. The oral compositions may include about 2,500 LU to about 8,000 LU lipase. The oral compositions may include about 50 HCU to about 250 HUC hemicellulase. The oral compositions may include one or more probiotic compounds, including but not limited to Lactobacillus acidophilus, Lactobacillus casei Lactobacillus plantarum, Lactobacillus rhamnosus, Bifidobacterium breve, and Bifidobacterium longum. In one example, the oral compositions include about 2,500,000,000 CFU of probiotics. The oral compositions may include stevia. In one example, the oral compositions include about 10 mg to about 20 mg stevia.

The present invention provides processes for preparing compositions for treatment of gastro-esophageal reflux disorders, which include incorporating at least two of: (i) one or more digestive enzymes; (ii) one or more probiotics; and (iii) stevia. The ingredients are admixed until a uniform mixture is obtained and thereafter the mixture is formed into suitable coating for an oral composition.

The present invention provides methods of treating a subject against a gastro-esophageal reflux disorder, which includes administering to a subject a therapeutically effective amount of the compositions of the present invention.

### DETAILED DESCRIPTION OF THE PRESENTLY PREFERRED EMBODIMENTS

The present invention provides compositions for treatment of a variety of gastro-esophageal reflux disorders, including gastro-esophageal reflux disease (GERD), acid indigestion, over-indulgence of food or drink, acid stomach, sour stomach, waterbrash/regurgitation, heartburn, such as episodic heartburn, nocturnal heartburn, and meal-induced heartburn, and dyspepsia. It is contemplated that the compositions of the present invention can also be used for treatment of a variety of functional gastrointestinal disorders, such as irritable bowel syndrome (IBS). A “functional disorder” refers to a “disorder of functioning” where the body's normal activities in terms of the movement of the intestines, the sensitivity of the nerves of the intestines, or the way in which the brain controls some of these functions is impaired.

“Treating” or “treatment” as used herein refers to the treating or treatment of a disorder, disease, or medical condition. Examples of a disorder, disease, or a medical condition are gastro-esophageal reflux disorders such as heartburn.

“Therapeutically effective amount” refers to an amount sufficient to effect treatment when administered to a subject. For example, therapeutically effective amount of a composition for treatment of a gastro-esophageal reflux disorder refers to the amount of active ingredients in the composition that will effectuate a therapeutic response in the treated subject against gastro-esophageal reflux disorder.

The present invention contemplates the use of one or more ingredients from each of at least two groups of compounds: 1) one or more digestive enzymes; 2) one or more probiotic compounds; and 3) stevia. These three groups of compounds (digestive enzymes, probiotics, and stevia) are referred to herein as “active ingredients” or “ingredients”. The synergistic effect of ingredients belonging to two or more of these groups of compounds results in relief from gastro-esophageal reflux disorders such as heartburn, acid reflux, and GERD, typically without the potential side effects of the pharmaceuticals used to treat these conditions.

In one aspect of the present invention, it is possible to treat a subject who has a gastro-esophageal reflux disorder with a combination of one or more digestive enzymes and one or more probiotic compounds (probiotics). In another aspect of the present invention, it is possible to treat a subject who has a gastro-esophageal reflux disorder with a combination of one or more digestive enzymes and stevia. In another aspect of the present invention, it is possible to treat a subject who has a gastro-esophageal reflux disorder with a combination of one or more probiotic compounds and stevia. Yet in another aspect of the present invention, it is possible to treat a subject who has a gastro-esophageal reflux disorder with a combination of one or more digestive enzymes, one or more probiotic compounds, and stevia.

### Digestive Enzymes

Digestive enzymes are enzymes in the alimentary tract that break down food components so that they can be taken up by the organism. The main sites of action of these enzymes are the oral cavity, the stomach, the duodenum and the jejunum. The digestive enzymes are secreted by different glands: the salivary glands, the glands in the stomach, the pancreas, and the glands in the small intestines. The enzymes that get secreted in the stomach are called gastric enzymes. Examples of these enzymes include: pepsin (a peptidase that breaks proteins into smaller peptide fragments), gelatinase (degrades type I and type V gelatin and type IV and V collagen), gastric amylase (degrades starch), gastric lipase (tributyrase), and others.

In one aspect, the present invention contemplates the use of digestive enzymes as one type of ingredient in the compositions of the present invention useful for the treatment of a variety of gastro-esophageal reflux disorders. For example, one dose (e.g., one tablet or one capsule) of the composition of the present invention may include one or more of: peptidase in the amount of about 5,000 HUT to about 30,000 HUT, and preferably in the amount of about 15,000 HUT; protease in the amount of about 15,000 HUT to about 50,000 HUT, and preferably about 29,000 HUT; bromelain in the amount of about 100 GDU to about 400 GDU, and preferably in the amount of about 180 GDU; papain in the amount of about 100,000 PU to about 200,000 PU, and preferably in the amount of about 150,000 PU; amylase in the amount of about 5,000 DU to about 25,000 DU, and preferably in the amount of about 11,750 DU; lactase in the amount of about 500 ALU to about 1,500 ALU, and preferably in the amount of about 875 ALU; lipase in the amount of about 2,500 LU to about 8,000 LU, and preferably in the amount of about 5,500 LU; and hemicellulase in the amount of about 50 HCU to about 250 HUC, and preferably in the amount of about 115 HCU. The abbreviations that follow the amounts of enzymes refer to standardized units of enzymatic activity. Various combinations of these and other digestive enzymes, including but not limited to cellulase, amyloglucosidase, maltase, ficin, disaccharidase, carboxypeptidase, sucrase, aminopeptidase, and chymotrypsin, can be used for practicing the invention, so long as the digestive enzymes help improve digestibility of food in the stomach.

In one example of the present invention, a composition comprising one or more digestive enzymes, in synergy with one or more probiotic compounds or stevia, or in synergy with both one or more probiotic compounds and stevia, results in relief from one or more gastro-esophageal reflux disorders such as heartburn, acid reflux, and GERD.

### Probiotics

Probiotics (probiotic compounds) are dietary supplements containing potentially beneficial bacteria or yeast. Lactic acid bacteria are the most common microbes used. Lactic acid bacteria have been used in the food industry for many years, because they are able to convert sugars (including lactose) and other carbohydrates into lactic acid.

The most common forms for probiotics are dairy products and probiotics-fortified foods. However, tablets and capsules containing the bacteria in freeze dried form are also available.

Some common probiotics useful for practicing the present invention include various species of the genera Bifidobacterium and Lactobacillus, such as: Bifidobacterium bifidum, Bifidobacterium breve, Bifidobacterium infantis, Bifidobacterium longum, Lactobacillus acidophilus, Lactobacillus bulgaricus, Lactobacillus casei, Lactobacillus fermentum, Lactobacillus helveticus, Lactobacillus plantarum, Lactobacillus reuteri, Lactobacillus rhamnosus, Lactobacillus salivarius, Lactobacillus GG, Lactococcus diacetylactis. Other species of bacteria can be used as probiotics, e.g. Pediococcus acidilactici, Bacillus coagulans, and Streptococcus thermophilus. Yeast, such as Saccharomyces boulardii, can also be used as a probiotic compound. Various combinations of these and other probiotic compounds can be used for practicing the invention, so long as the probiotic compounds help improve digestibility of food in the stomach. The amount of probiotic compounds can vary. For example, one dose (e.g., one tablet or one capsule) of the composition of the present invention preferably includes about 2,500,000,000 CFU (colony forming units) of probiotic compounds (e.g. bacteria).

In one aspect, the present invention contemplates the use of probiotic compounds such as one or more cultures of Lactobacillus acidophilus, Lactobacillus casei Lactobacillus plantarum, Lactobacillus rhamnosus, Bifidobacterium breve, and Bifidobacterium longum. In one example of the present invention, a composition comprising one or more probiotic compounds, in synergy with one or more digestive enzymes or stevia, or in synergy with both one or more digestive enzymes and stevia, results in relief from gastro-esophageal reflux disorders such as heartburn, acid reflux, and GERD.

### Stevia

For purposes of this invention, “stevia” refers to the extract of Stevia (also called sweet leaf or sugar leaf), which is a genus of about 150 species of herbs and shrubs in the Asteraceae family. In particular, for purposes of this invention, “stevia” refers to the extract of Stevia rebaudiana. Stevia extract is sometimes called stevioside. With its extracts having up to 300 times the sweetness of sugar, stevia has garnered attention with the rise in demand for low-carbohydrate, low-sugar food alternatives. Stevia also has shown promise in medical research for treating such conditions as obesity and high blood pressure. Stevia has negligible effect on blood glucose, and is attractive as a natural sweetener to diabetics and others on carbohydrate-controlled diets.

Health and political controversies have limited stevia's availability in many countries. For example, stevia is widely used as a sweetener in Japan. In the USA and Canada stevia is available as a dietary supplement, although not as a food additive.

In one aspect, the present invention contemplates the use of stevia (i.e., stevia extract). In one example of the present invention, a composition comprising stevia, in synergy with one or more digestive enzymes, one or more probiotic compounds, or in synergy with both one or more digestive enzymes and one or more probiotic compounds, results in relief from gastro-esophageal reflux disorders such as heartburn, acid reflux, and GERD. The amount of stevia can vary. For example, one dose (e.g., one tablet or one capsule) of the composition of the present invention may include about 10 mg to about 20 mg of stevia. In one example, one dose of the composition of the present invention preferably includes about 15 mg of stevia.

### Formulations

The compositions of the present invention are preferably systemically absorbable by a human. The compositions of the present invention are preferably delivered to a subject orally. Accordingly, the present invention provides methods of producing tablets, capsules, powder, drops, and other products that comprise the compositions of the present invention and that can be delivered orally. The powder can be mixed in water or other consumable liquid and may be flavored for taste. Examples of methods and compositions for feeding mammals are shown, e.g., in United States Patent Application Publication No. US 2004/0156882 A1, incorporated by reference herein.

In one embodiment, the formulations suitable for oral delivery are prepared by forming a base solution that includes edible film forming agents, such as maltodextrins, hydrocolloids and fillers and processing the base solution to form an edible film that is suitable for coating of, impregnating of, or admixing with, various compositions of this invention. Typically, the base solution of such a film is prepared by adding an initial mixture of dry ingredients to water that is stirred.

Additional ingredients, such as flavor/emulsifier blends, sweeteners, softeners, color, the like or combinations thereof, can be added to the base solution. In one aspect, the solution is stirred continuously and heated at a temperature ranging from about 40° C. to about 60° C. The solution can then be dried in any suitable manner, thereby forming an edible film that includes the compositions of the present invention. It should be appreciated that any suitable type, number and arrangement of process procedures or steps (i.e., mixing, heating, drying, cooling, and addition of ingredients), process parameters (i.e., temperature, pressure, pH, process times) or the like can be utilized. Examples of oral delivery systems for functional ingredients such as drugs, nutritional supplements, botanicals, and vitamins, are shown, e.g., in United States Patent Application Publication No. US 2005/0208141A1, incorporated by reference herein. As well, examples of nutritional systems, albeit for nervous system disorders, are shown in U.S. Pat. No. 6,399,114 B2, incorporated by reference herein.

In one aspect, controlled release of the compositions of the present invention is preferred. When such formulation is desired, any relevant controlled formulation technique for preparing an oral composition with controlled release may be applied. Thus, the dosage form may be in the form of a liquid having particles dispersed in a dispersion medium or it may be in the form of a single or a multiple unit dosage form intended for use as such as for dispersing in a dispersion medium before use.

It may be desirable to provide fast release of the compositions, in order to provide fast relief from symptoms. Using the compositions described herein, a person skilled in the art will know how to incorporate a part that gives rise to a relatively fast release of the active ingredient. As an example, such a part may be incorporated in an outermost coating layer comprising the active ingredient, or it may be incorporated in the form of pellets formulated without retarding agents neither in the cores nor in a coating.

For any use that requires fast release of compositions, the particle size reduction is essential to see the full benefit of the active ingredient. For many active ingredients, there is a critical level required to obtain a response. Thus it is essential that at least an effective amount of the active ingredient be in small particle form. Effective amounts depend upon the ingredients used (i.e., digestive enzymes, probiotic compounds, stevia) and the end result desired. For example, one or more ingredients may be added to a pill, capsule, or drop coating, which is a water soluble matrix, such that during the chewing period, the ingredients may be released quickly, resulting in a fast release. For instance, U.S. Pat. No. 6,645,535, incorporated by reference herein, discloses a coating made with a syrup having an antacid dispersed therein, resulting in a fast release of the antacid.

Examples of different controlled release technologies are: single units based on coated matrix, double or triple compression, or multilayer coating; and multiple units including units having a controlled release coating, units having a controlled release matrix, units having a controlled release compression coating, and units with a multilayer coating. Other examples include liquid fill compositions with modified release components, e.g., as described in United States Patent Application Publication No. US 2007/0003663 A1, incorporated by reference herein.

In one aspect of the invention, coated matrix technology is used to coat a sparingly soluble and/or swellable polymer, in which two or more active ingredients are embedded, with an insoluble diffusion barrier. The diffusion of active ingredients is controlled by the matrix and the coat. It is possible to use an outer film layer containing active ingredients, which is applied on the coated matrix. Alternatively, enteric coated units can be embedded in the matrix.

In another aspect of the invention, a formulation based on double or triple compression contains a core of a polymer having active ingredients incorporated. This core is compression-coated with a polymer with active ingredients incorporated in the same or another concentration than in the core. When triple compression is employed, the coated core is compression-coated once more with a polymer with active ingredients in the same or another concentration as in the first coat. Finally, the double or triple compression unit is spray-coated and the active ingredients are incorporated in the coat. However, the concentrations of active ingredients in the different coats may vary markedly. When the active ingredient of the first layer has been almost depleted, the next layer takes over and levels out or changes the release profile.

In a multilayer coating formulation, an inert core is coated with several layers of diffusion barriers, each barrier containing different concentrations of active ingredients. The concentration should be highest in the inner coat and lowest in the outer coat. The purpose of the concentration gradient is to compensate for the increasing diffusion distance closer to the core. The thickness of the diffusion barriers and the concentration gradients need to be correctly adjusted. The multilayer technologies might be optimized by the use of an enteric polymer, and/or by the use of an amylose containing film coating such as a coating containing ethylcellulose and amylose. Furthermore, spray coating with active ingredients gives an immediate burst of the ingredients.

Multiple unit systems may be used, comprising chewable pellets, granules, crystals, mini tablets or mixtures thereof. In such systems, some units may be uncoated, whereas other units may be formulated as a matrix or a coated matrix. The units can be compressed. The active ingredients may also be present in the composition in the form of a multiplicity of individual units such as, for example chewable pellets, minitablets, and crystals of active substances. The two parts may be in admixture, or they may comprise at least two different types of chewable pellets, minitablets, or crystals, the first type of pellets corresponding to the first part and the second type of pellets corresponding to the second part. Alternatively, release of active ingredients according to the invention may also be obtained if individual units contain relatively large crystals of the active drug substance. In such cases, the unit size is typically in the micrometer range.

The release of active ingredients may be achieved with any compound which is a natural fast release compound, or it may be a compound which has been treated such that it will possess release properties during chewing and/or dissolution. Treating methods contemplated include encapsulation, co-drying and dissolution of the active ingredients into various solvents including water, alcohols, flavors, and the like.

In another embodiment of the invention, desired effect may be obtained when the active ingredients are encapsulated within a biodegradable-biocompatible polymeric matrix, according to many of the microencapsulation teachings in the art. The microcapsules may be comprised of a core of polypeptide or other biologically active agent encapsulated in a matrix of poly(lactide/glycolide) copolymer.

In one embodiment of the invention, two or more active ingredients are mixed with a pharmaceutically acceptable carrier. A “pharmaceutically acceptable carrier” means any conventional pharmaceutically acceptable carrier, vehicle, or excipient that is used in the art for production and administration of pharmaceutical products. Pharmaceutically acceptable carriers are typically non-toxic, inert, solid or liquid carriers.

### Administration

The compositions of the present invention can be administered in a variety of ways. Preferably, the compositions are administered orally, e.g. in the form of pills, tablet, drops, or capsules.

The frequency of administration is determined upon consideration of the type and amount of ingredients used, and the severity of the gastro-esophageal reflux disorder. The compositions of the present invention can be used as a dietary supplement. For example, if the composition of the present invention is manufactured in the form of a tablet for oral administration, one tablet can be consumed, e.g. chewed, three times a day, with meals.

### EXAMPLES

In one example, the following ingredients are used in a tablet suitable for oral administration: 1) digestive enzymes, amount per tablet: peptidase 15,000 HUT; protease 29,000 HUT; bromelain 180 GDU; papain 150,000 PU; amylase 11,750 DU; lactase 875 ALU; lipase 5,500 LU; hemicellulase 115 HCU; 2) non-dairy probiotic blend: 2,500,000,000 CFU (colony forming units) per tablet, containing Lactobacillus acidophilus, Lactobacillus casei Lactobacillus plantarum, Lactobacillus rhamnosus, Bifidobacterium breve, and Bifidobacterium longum; and 3) stevia, 15 mg per tablet. The tablet may optionally contain other ingredients such as xylitol, orange powder, carrot powder, citric acid, natural orange flavor, vegetable stearate, etc. One tablet can be consumed, e.g. chewed, three times a day, with meals.

The inventors have already produced such chewable tablets that include a synergistic combination of three types of compounds as indicated above, and these tablets are being sold as dietary supplements under the name of Homefirst® Natural Chewable Digestive Enzymes with Probiotics. This novel synergistic combination works better than any of the individual ingredients for purposes of relieving acid reflux, heartburn, GERD, and related gastro-esophageal reflux disorders.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters normally encountered in medical prevention and therapy, obvious to those skilled in the art, are within the scope of this invention. All publications, patents, and patent applications cited herein are incorporated by reference in their entirety for all purposes.

What is claimed is:

1. A composition for treatment of a gastro-esophageal reflux disorder, comprising at least two of: (i) one or more digestive enzymes; (ii) one or more probiotics; and (iii) stevia.

2. The composition of claim 1 wherein the one or more digestive enzymes comprises one or more of peptidase, protease, bromelain, papain, amylase, lactase, lipase, and hemicellulase.

3. The composition of claim 1 further comprising about 5,000 HUT to about 30,000 HUT peptidase.

4. The composition of claim 1 further comprising about 15,000 HUT to about 50,000 HUT protease.

5. The composition of claim 1 further comprising about 100 GDU to about 400 GDU bromelain.

6. The composition of claim 1 further comprising about 100,000 PU to about 200,000 PU papain.

7. The composition of claim 1 further comprising about 5,000 DU to about 25,000 DU amylase.

8. The composition of claim 1 further comprising about 500 ALU to about 1,500 ALU lactase.

9. The composition of claim 1 further comprising about 2,500 LU to about 8,000 LU lipase.

10. The composition of claim 1 further comprising about 50 HCU to about 250 HUC hemicellulase.

11. The composition of claim 1 wherein the one or more probiotics comprises one or more of Lactobacillus acidophilus, Lactobacillus casei, Lactobacillus plantarum, Lactobacillus rhamnosus, Bifidobacterium breve, and Bifidobacterium longum.

12. The composition of claim 1 further comprising about 2,500,000,000 CFU of probiotics.

13. The composition of claim 1 further comprising about 10 mg to about 20 mg stevia.

14. The composition of claim 1 wherein the gastro-esophageal reflux disorder is a gastro-esophageal reflux disease (GERD), acid indigestion, over-indulgence of food or drink, acid stomach, sour stomach, waterbrash/regurgitation, heartburn, or dyspepsia.

15. An oral composition for treatment of a gastro-esophageal reflux disorder, comprising:
a) a vehicle for oral delivery;
b) a water soluble portion; and
c) a coating layer including at least two of: (i) one or more digestive enzymes; (ii) one or more probiotics; and (iii) stevia.

16. The oral composition of claim 15 wherein the one or more digestive enzymes comprises one or more of peptidase, protease, bromelain, papain, amylase, lactase, lipase, and hemicellulase.

17. The oral composition of claim 15 further comprising about 5,000 HUT to about 30,000 HUT peptidase.

18. The oral composition of claim 15 further comprising about 15,000 HUT to about 50,000 HUT protease.

19. The oral composition of claim 15 further comprising about 100 GDU to about 400 GDU bromelain.

20. The oral composition of claim 15 further comprising about 100,000 PU to about 200,000 PU papain.

21. The oral composition of claim 15 further comprising about 5,000 DU to about 25,000 DU amylase.

22. The oral composition of claim 15 further comprising about 500 ALU to about 1,500 ALU lactase.

23. The oral composition of claim 15 further comprising about 2,500 LU to about 8,000 LU lipase.

24. The oral composition of claim 15 further comprising about 50 HCU to about 250 HUC hemicellulase.

25. The oral composition of claim 15 wherein the one or more probiotics comprises one or more of Lactobacillus acidophilus, Lactobacillus casei Lactobacillus plantarum, Lactobacillus rhamnosus, Bifidobacterium breve, and Bifidobacterium longum.

26. The oral composition of claim 15 further comprising about 2,500,000,000 CFU of probiotics.

27. The oral composition of claim 15 further comprising about 10 mg to about 20 mg stevia.

28. The oral composition of claim 15 wherein the gastro-esophageal reflux disorder is a gastro-esophageal reflux disease (GERD), acid indigestion, over-indulgence of food or drink, acid stomach, sour stomach, waterbrash/regurgitation, heartburn, or dyspepsia.

29. A process for preparing a composition for treatment of a gastro-esophageal reflux disorder which comprises incorporating at least two of: (i) one or more digestive enzymes; (ii) one or more probiotics; and (iii) stevia, admixing the ingredients until a uniform mixture is obtained and thereafter forming the mixture into a composition suitable for administration to a subject with gastro-esophageal reflux disorder.

30. The process of claim 29 wherein the mixture can be formed into a coating for an oral composition.

31. The process of claim 29 wherein the composition suitable for administration to a subject with gastro-esophageal reflux disorder is a dietary supplement.

32. A method of treating a subject against a gastro-esophageal reflux disorder, which comprises administering to the subject a therapeutically effective amount of the composition of claim 1.

33. A method of treating a subject against a gastro-esophageal reflux disorder, which comprises administering to the subject a therapeutically effective amount of the oral composition of claim 15.
